(19) United States
(12) Patent Application Publication
(10) Pub. No.: US 2013/0303956 A1
(43) Pub. Date: Nov. 14, 2013

(54) LUMBAR SUPPORT BELT

(71) Applicants: Gerard Anglada, Saint Etienne (FR); Frederic Girard, Paris (FR)

(72) Inventors: Gerard Anglada, Saint Etienne (FR); Frederic Girard, Paris (FR)

(73) Assignee: Gibaud, Saint Etienne (FR)

(21) Appl. No.: 13/981,452

(22) PCT Filed: Jan. 3, 2012
(86) PCT No.: PCT/FR2012/050016

§ 371 Date: Jul. 24, 2013

(30) Foreign Application Priority Data
Jan. 31, 2011 (FR) 11/50738

(51) Int. Cl. A61F 5/02

(52) U.S. Cl. CPC A61F 5/028
USPC 602/19

(57) ABSTRACT

A lumbar support belt has a dorsal plate, first and second half-belts, and a device for adjusting the pressure exerted by the belt on the dorsolumbar region of the patient. The dorsal plate has lateral extensions including a lower edge shaped to bear against the patient's iliac crests. The device includes first and second tightening straps secured to a distal end of a respective half-belt and received through a respective second free buckle. The first and second tightening straps are intended to be stacked on the lateral extensions of the plate against the patient's lumbar-abdominal region.

The invention relates to a lumbar support belt.

The invention more generally relates to the field of dorsal orthopedics.

Pathologies frequently observed in patients include lumbar arthrosis, lumbar pain, and lumbago. In order to soothe patients suffering from these pathologies, those patients are advised to wear a lumbar support belt. A lumbar support belt is a medical belt made from an elastic textile material, the purpose of which is to provide maintenance of the lumbar region as well as the abdominal region for the patient wearing it. Wearing a lumbar belt in particular provides a posture reminder to decrease pain and ease the contracted muscles.

A known lumbar support belt by the applicant is for example described in patent application U.S. Pat. No. 6,322,529.

One inherent drawback of the lumbar belt described in the aforementioned document is that the shape of the dorsal plate does not allow precise positioning of the lumbar belt on the patient. In particular, during placement of the belt, the dorsal plate frequently rotates slightly along an axis perpendicular to the frontal plane of the patient. The dorsal plate is then off-centered with respect to the middle axis of that patient.

Poor positioning of the belt is detrimental insofar as it may result in accentuating a posture defect in a patient and can also aggravate the pathology from which the patient suffers. The invention thus aims to provide a lumbar support belt that is easy to position.

Furthermore, in order for a lumbar support belt to produce an antalgic effect, the pressure exerted by the belt on the patient's dorsal-lumbar area must be sufficient. To that end, the patient must often exert a significant force to place the belt.

However, patients suffering from lumbar arthrosis generally also have arthrosis of the hands. These patients with arthrosis of the hands experience great difficulty in producing the force necessary to place the belt appropriately.

In order to offset this drawback, the belt described in the aforementioned document comprises a half-turn system to reduce the force exerted by the patient. However, the force required from the patient remains significant.

The invention aims to provide a lumbar support belt producing significant compression strength with minimum exertion for the patient.

The invention thus relates to a lumbar support belt, including:
a dorsal plate designed to bear against the patient's dorsal-lumbar area;
first and second half-belts designed to bear against the patient's lumbar-abdominal area, said first and second half-belts being slidingly mounted with the plate;
a device for adjusting a pressure exerted by the belt on the patient's dorsal-lumbar area, said device comprising:
   first buckles fastened on the proximal ends of the first and second half-belts;
   second buckles that are free with respect to the proximal ends of the first and second half-belts;
   a dorsal strap received through said buckles forming a zigzag mesh.

Furthermore, according to a general definition of the invention:
the dorsal plate includes lateral extensions comprising a lower edge configured to bear against the patient's iliac crests, and
the device also comprises first and second tightening straps fastened on the one hand on a distal end of one respective half-belt, and on the other hand received through a second respective free buckle, the first and second tightening straps being designed to be superimposed on the lateral extensions of the plate against the patient's lumbar-abdominal area.

The lumbar belt according to the invention is advantageous in that the lateral extensions of the dorsal plate make it possible to position the dorsal plate on the patient precisely, and thereby make it possible to position the lumbar support belt precisely. In fact, when the patient places the belt, he places the lower edges of the lateral extensions bearing directly against the iliac crests. The patient thus has fixed references (i.e., the iliac crests) allowing excellent repeatability of the positioning of the belt, and limiting the risk of poor positioning of said belt.

Due to the presence of the lateral extensions, the dorsal plate is not merely a dorsal support device, but also allows bearing on the “soft” lateral areas of the patient, favoring gripping of the waist.

The tightening straps facilitate the actuation of the half-turn system and make it possible to still further decrease the force exerted by the patient. One advantage of the straps passing through buckles with respect to threads passing through the groove of pulleys is that the risk of breaking or disengagement outside the buckles is considerably reduced. Furthermore, the thickness of the system is reduced.

Lastly, once the pressure is adjusted, the tightening straps are superimposed on the lateral extensions of the dorsal plate against the patient's lumbar-abdominal area. Such a configuration of the tightening straps causes the deformation of the dorsal plate, which assumes an “hourglass” shape. This deformation produces a thrust force between the iliac crests and the rib cage, in a direction parallel to the middle axis of the patient. Thus, the burden borne by the lumbar L5 and sacral S1 vertebrae is minimized. The compression strength of the belt is thus improved.

The embodiments of this invention may include the features below.

The dorsal plate may include a central opening for freeing the patient's posterior processes, the majority of said central opening extending along an axis designed to be positioned in the sagittal plane of the patient.

The dorsal plate thus configured is advantageous in that the presence of the central opening does not hinder the spines of vertebra of the patient's spine when the dorsal plate deforms. Thus, the belt makes it possible to adapt to the morphology of each patient.

The dorsal plate may comprise a scoring extending along an axis designed to be positioned in the sagittal plane of the patient.

The dorsal plate thus configured is advantageous in that the scoring guides the deformation of the dorsal plate and still further minimizes the force necessary to deform the dorsal plate.

The ends of the free parts of the tightening straps may also form a loop to receive one of the patient's fingers, so as to facilitate the exertion of a pulling force on the tightening straps by the patient.

Furthermore, the proximal ends of the half-belts may each include at least one loop inside which a lateral extension of the dorsal plate is received, so as to allow sliding of the half-belts with the dorsal plate.

Under these conditions, the mounting and alternately disassembly of the half-belts and the dorsal plate is made easier.

Lastly, the belts may comprise first and second elastic return means connecting the dorsal plate on the one hand and the first or second half-belt, respectively, on the other hand, so as to continuously keep the plate and the half-belts centered with respect to one another. Means are sewn on the dorsal plate and are fastened on the corresponding half-belt by self-fastening pieces of fabric.

The invention will be better understood upon reading the following description, provided solely as a non-limiting example and done in reference to the appended drawings, in which:

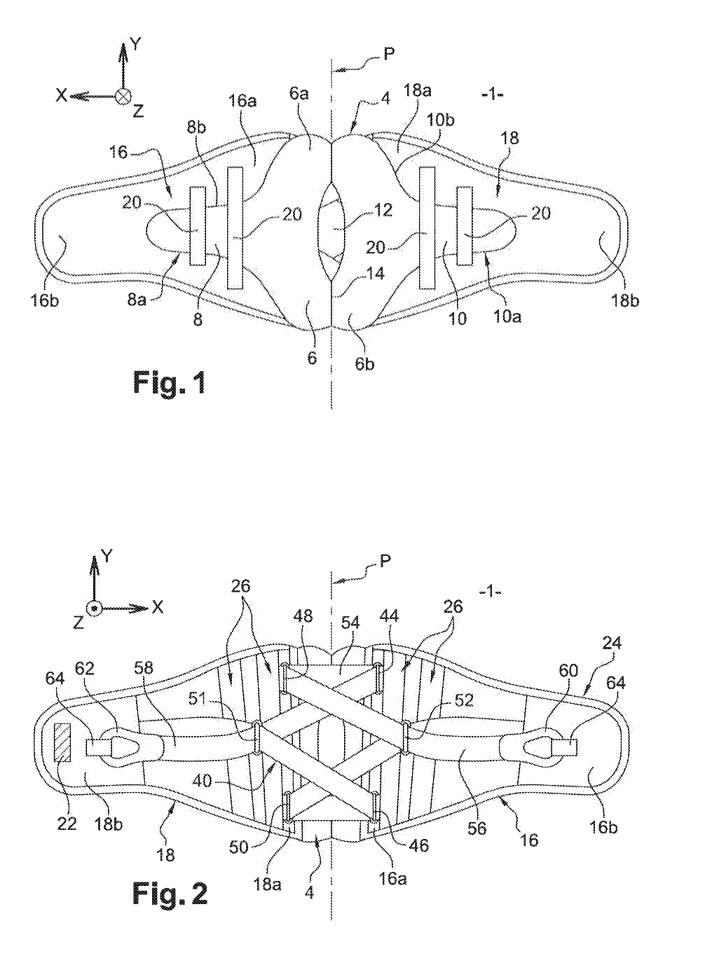

FIG. 1 is a diagrammatic illustration of an inner face of a lumbar support belt according to the invention,

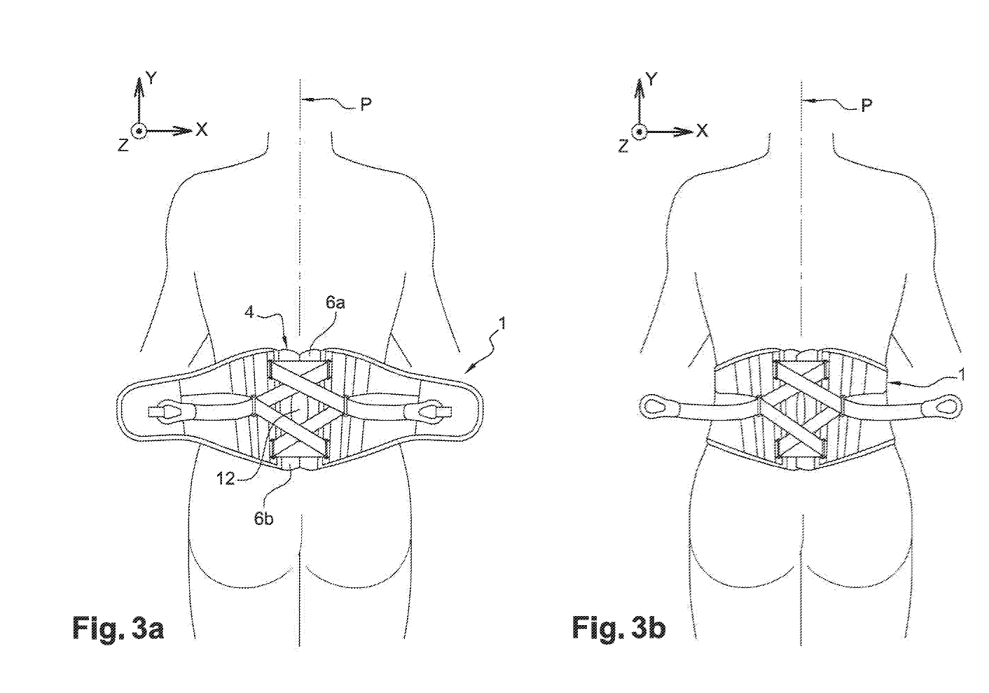

FIG. 2 is a diagrammatic illustration of an outer face of the belt of FIG. 1,

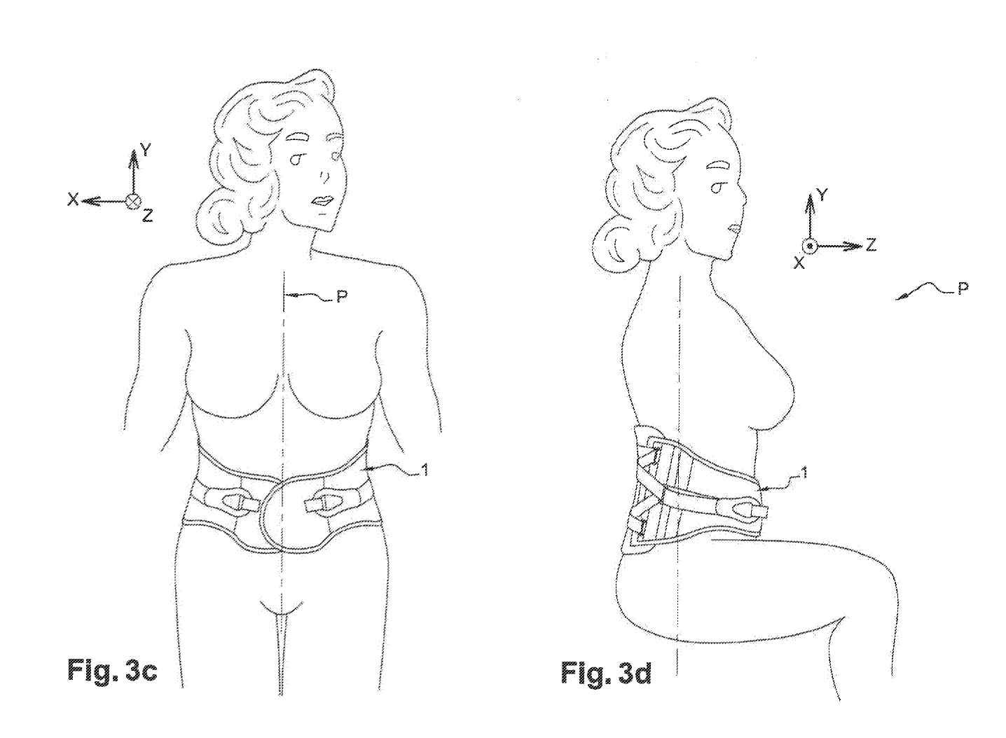

FIGS. 3a and 3b are diagrammatic illustrations, in back view, of a patient wearing the lumbar support belt of FIGS. 1 and 2, and

FIGS. 3c and 3d are front and side view, respectively, diagrammatic illustrations of the patient wearing the lumbar support belt of FIGS. 1 and 2.

These figures are oriented using a same orthogonal reference formed by axes X, Y and Z perpendicular to each other.

FIG. 1 illustrates a lumbar support belt 1. The belt 1 includes:
a rigid dorsal plate 4,
half-belts 16 and 18, and
a device 40 to adjust the setting of the belt 1 (better shown in FIG. 2).

The dorsal plate 4 can be made from an open structure high-density polyethylene foam. This material makes it possible to limit the maceration effect during use of the belt. This foam is tufted with a loop-pile fabric in contact with the user's back. The dorsal plate may also be made from a thermoformable plastic (with a low application temperature). It may be modeled after the shape of the user's back.

The dorsal plate 4 is formed by a patch 6 and lateral extensions 8 and 10. The dorsal plate 4 is preferably formed in a single piece.

In the illustrated example, the patch 6 is generally oval-shape. The patch 6 comprises a central opening 12 passing all the way through it. In the example, the central opening 12 extends primarily along an axis parallel to the axis Y. Here, the transverse cross-section of the opening 12 has an oval shape.

The patch 6 comprises a scoring 14. In the example, the scoring 14 extends along an axis parallel to the axis Y.

The extensions 8 and 10 extend on either side of the patch 6 along the axis X. The extensions 8 and 10 have a flared shape, the width of the extensions 8 and 10 at their respective distal end being smaller than the width of said extensions 8 and 10 at the junction with the patch 6. The width of the extensions 8 and 10 is measured parallel to the axis Y. The extensions 8 and 10 comprise lower edges 8a and 10a, respectively, and upper edges 8b and 10b, respectively.

In the rest of this description, the “centering plate of the dorsal plate 4” refers to a plane forming a plane of symmetry for that plate 4, said plane being parallel to the axes Y and Z. In the example, the centering plane is combined with a plane P. Here, this plane passes through the axis along which the scoring 14 extends.

The half-belts 16 and 18 can be made from an elastic fabric with a force greater than 350 cN per centimeter at 30% extension. Preferably, the fabric is openworked so as to favor ventilation.

The half-belts 16 and 18 respectively comprise:
proximal ends 16a and 18a, and
distal ends 16b and 18b.

The half-belts 16 and 18 have a flared shape, the width of the extensions of the half-belts 16, 18 at their distal ends 16b and 18b being smaller than the width of said half-belts 16 and 18 at their proximal ends 16a and 18a. The width of the half-belts 16 and 18 is measured parallel to the axis Y.

The half-belts 16 and 18 are slidingly mounted with the dorsal plate 4. To that end, the proximal ends 16a and 18a include loops 20 inside which the lateral extensions 8 and 10 are received. In the example, the proximal ends 16a and 18a each comprise two loops 20. Here, these loops 20 are sewn on the inner face of the half-belts 16 and 18.

The description henceforth refers to FIG. 2.

The distal ends 16b and 18b comprise closing means for the belt 1. In this example, the closing means are formed by textile strips 22 and 24 with loops and hooks, respectively. Here, the textile strips 22 and 24 are respectively sewn on the inner face of the distal end 16b and on the outer face of the distal end 18b.

The half-belts 16 and 18 preferably comprise sheaths 26 inside which stays are housed. In the example, each half-belt 16 and 18 comprises two sheaths 26 formed at the proximal ends 16a and 18a. The stays are for example made from spring steel.

In the continuation of this description, the “centering plane of the half-belts 16 and 18” refers to a plane forming a plane of symmetry for said half-belts 16 and 18, said plane being parallel to the axes Y and Z. Here, this centering plane is combined with the plane P.

The belt 1 advantageously includes elastic return straps (not shown) to continuously keep the plate 4 and the half-belts 16, 18 centered with respect to one another.

“Keeping the dorsal plate 4 and the half-belts 16 and 18 centered with respect to one another” means keeping the centering planes of the plate 4 and the half-belts 16 and 18 combined.

The return straps are fastened on the one hand to the outer face of the patch 6 and on the other hand to the inner face of the respective half-belts 16, 18. As an example, here the return straps are sewn to the patch 6 on the one hand. On the other hand, the return straps comprise a textile strip with loops to be fastened to a strip with hooks sewn on the inner face of their respective half-belt 16, 18.

The half-belts 16 and 18 are mechanically connected to each other by the device 40. The device 40 forms a half-turn system comprising:
buckles 44, 46 fastened on the proximal end 16a,
buckles 48, 50 fastened on the proximal end 18a,
buckles 51, 52 that are free with respect to the proximal ends 16a and 18a,
a dorsal strap 54 received through the buckles 44, 46, 48, 50, 51 and 52 forming a zigzag mesh, and
tightening straps 56, 58.

The tightening straps 56, 58 comprise an end fastened to the distal ends 16b, 18b, respectively, and a free end. The straps 56, 58 are received through the buckles 52 and 51, respectively.

Preferably, the straps 54, 56 and 58 are made from a non-extendable fabric.

In the example, the free ends of the straps 56, 58 comprise loops 60 and 62, respectively, to receive the patient's finger. Consequently, the diameter of said rings 60, 62 is preferably greater than 3 cm, 5 cm or 10 cm. The rings 60, 62 may be provided with textile fasteners that protect the commissure during pulling and allow comfortable and intuitive pushing.

The free ends of the straps 56, 58 comprise means 64 for fastening, and alternatively releasing, the straps 56, 58 of the half-belts 16. In the example, the means 64 are textile strips with loops that can be fastened to strips (not shown) with hooks sewn on the outer faces of half-belts 16, 18.

The implementation of the belt 1 is now described in reference to FIGS. 3a, 3b, 3c and 3d.

In the rest of this description, the “middle axis” of the patient defines the axis passing through the apex of the patient's skull and the patient's center of gravity. In the example, the middle axis is parallel to the axis Y.

The “sagittal plane” of the patient refers to a plane parallel to the middle axis and passing through a point situated midway between the patient's eyes. Here, the sagittal plane is parallel to the axes Y and Z.

During the first step shown in FIG. 3a, the patient positions the belt 1. To that end, the patient grasps the belt 1 using the distal ends 16b and 18b and positions the patch 6 of the plate 4 bearing against the dorsal-lumbar area. The patch 6 is positioned such that:
an upper part 6a of the patch 6 bears against the patient's dorsal vertebra D11,
a lower part 6b bears against the patient's sacral vertebra S2, and
the opening 12 is placed across from the patient's posterior processes.

At the same time, the patient positions the lateral extensions 8 and 10 bearing against the lumbar-abdominal area such that the lower edges 8a and 10a bear against the iliac crests.

In this position, the centering plane of the plate 4 is combined with the sagittal plane of the patient. Due to the presence of the elastic return straps, the centering plane of the half-belts 16 and 18 is also combined with the sagittal plane of the patient.

During a second step illustrated in FIG. 3b, the patient closes the belt 1. To that end, the patient presses the half-belts 16 and 18 against the lumbar-abdominal area such that:
the proximal ends 16a and 18a bear against the patient's dorsal-lumbar area, and
the distal ends 16b and 18b bear on one another against the patient's abdomen.

Under these conditions, the textile strips 22 and 24 with loops and hooks, respectively, close the belt 1.

Lastly, during a third step illustrated in FIGS. 3c and 3d, the patient adjusts the pressure exerted by the belt 1 in the dorsal-lumbar area using the device 40.

To that end, the patient grasps the straps 56, 58 using the loops 60 and 62.

The patient then exerts a pulling force on the straps 56, 58 while bringing the free ends of the straps 56, 58 toward his abdomen. In response to the exertion of that pulling force, the buckles 51, 52 tend to move away from each other.

Under these conditions, the strands of the strap 54 situated between the buckle 51 and the buckles 44, 56 become longer. Likewise, the strands of the strap 54 situated between the buckle 52 and the buckles 48, 50 become longer.

In response, the strands of the strap 54 situated between the buckles 44 and 48, and between the buckles 46 and 50, become shorter. Thus, as the patient exerts the pulling force on the straps 56 and 58, the proximal ends 16a and 18a come closer to each other.

The device 40 thus makes it possible to convert a pulling force exerted by the patient into a pressing force exerted by the belt 1 on the dorsal-abdominal area of the patient.

The patient exerts the pulling force on the straps until reaching a desired pressure force exerted by the belt 1 on the dorsal-lumbar area. Once this pressure force is reached, the patient superimposes straps 56 and 58 with the lateral extensions 8 and 10 of the plate 4 against his lumbar-abdominal area.

Lastly, the patient fastens the straps 56, 58 on the half-belts 16 and 18 using means 64. Under these conditions, the straps 56, 58 deform the plate 4. In particular, the straps 56, 58 hollow out the dorsal and lateral parts of the plate 4, giving said plate 4 an hourglass shape. The circular pressure exerted by the tightening straps 56 and 58 on the patient's waist is converted into a pushing force on the iliac crests and the rib cage. This mechanical action bears part of the weight of the thorax, thereby lessening the burden on the lower vertebrae (L5, S1).

The flared shape of the half-belts 16 and 18 is advantageous insofar as it allows the half-belts 16 and 18 not to cover the patient's floating ribs. “Floating ribs” refer to ribs K11 and K12 of the patient. Patient comfort is thus maximized.

Furthermore, the lower edges of the half-belts 16 and 18 are significantly indented above the thigh (inguinal ligament) and allow the patient to assume a comfortable seated position without compression of the patient's epigastric area. Thus, the half-belts 16 and 18 avoid exerting stomach compression and stress on the patient's diaphragm.

The loops 60, 62 facilitate gripping of the straps 56, 58 and the exertion of a pulling force on said straps 56, 58 by a patient in a direction parallel to the axis X.

The mode of action of the belt according to the invention and the dorsal-lumbar maintaining plate is as follows.

The compression of the lumbar-abdominal area situated between the iliac crests and the floating ribs is done by the accumulation of the following compression elements:
the dorsal plate 4;
the extendable belt made up of two half-belts 16, 18;
two non-extendable pressure adjustment straps 56, 58 that pass over the lateral extensions 8, 10 of the dorsal plate 4.

During tightening of the straps 56, 58, the two half-belts 16, 18 tighten in the back owing to the device 40, the distance—in X—between them decreasing. This device 40 makes it possible to multiply the force exerted by the patient by three times, since the buckles 44, 51, 46 or 48, 52, 50 offer three multiplication points. The dorsal plate 4 slides in the loops 20, thereby avoiding uncomfortable folds.

The pressure exerted by the belt is essentially dorsal, first, before the two half-belts are attached to each other over the patient's abdominal area.

At the same time, the straps 56, 58 bear on the lateral extensions 8, 10, causing a deformation of the dorsal plate 4. The latter assumes the form of an hourglass or saddle, with a double curve allowing it both to hug the patient's lordosis and surround the patient's back and ribs. The belt thus adapts to the morphology of each patient. When the belt is tightened, the width of the opening 12—in direction X—is decreased. Very significant tightening may even lead to the opening 12 no longer being visible, its two substantially vertical edges extending closer together until they are substantially joined. The pulling of the belt tightening the latter makes it possible to tighten the waist of the dorsal plate 4 and the patient, without, however, bothering the spines of vertebra of the spine.

The geometric shape in the form of an hourglass, applied on the lumbar-abdominal segment, converts the circular pressure into pushing on the iliac crests and the rib cage. This mechanical action bears part of the weight of the thorax, thereby lessening the load on the lower segments: L5/S1 and L4/L5.

The sliding dorsal plate 4 provides both a connection and cohesion between the two half-belts 16, 18 and makes it possible to avoid the discomfort related to folding of those half-belts on the skin or clothing.

When the pulling force is released on the two half-belts, the elastic return means connecting the dorsal plate and one of the half-belts relax symmetrically, such that the centering of the dorsal plate 4 is preserved.

Lastly, the belt may comprise first and second elastic return means on the one hand, and the first or second half-belt on the other hand, respectively, to continuously keep the plate and the half-belts centered with respect to one another. Means are sewn on the dorsal plate and are fastened on the corresponding half-belt by pieces of self-fastening fabric.

Many other embodiments are possible.

In particular, it is possible to equip the lumbar support belt according to the invention with a pair of shoulder straps. For example, the shoulder straps comprise an end fastened on an upper part of the dorsal plate and a free end. Preferably, the shoulder straps cross for the first time on the patient's back, pass over the respective shoulders, pass under the axillae directly below those shoulders, cross a second time on the patient's back, and are fastened at the abdomen. Such shoulder straps make it possible to straighten the patient's shoulder, and are particularly suitable for patients suffering from kyphosis.

The number of buckles fastened to the proximal ends of the half-belts may be different from two.

The buckles may be replaced by eyelets or any other similar element.

The invention is of course not limited to the embodiments described above as examples, but on the contrary encompasses all technical equivalents and alternatives of the means described as well as combinations thereof.

1-6. (canceled)

7. A lumbar support belt, comprising:
a dorsal plate arranged to bear against a patient's dorsal-lumbar area;
first and second half-belts arranged to bear against the patient's lumbar-abdominal area, said first and second half-belts slidingly mounted with the plate, the dorsal plate including lateral extensions having a lower edge configured to bear against the patient's iliac crests; and
a device for adjusting a pressure exerted by the belt on the patient's dorsal-lumbar area, said device including:
first buckles fastened on the proximal ends of the first and second half-belts;
second buckles free with respect to the proximal ends of the first and second half-belts;
a dorsal strap received through said buckles forming a zigzag mesh; and
first and second tightening straps fastened on a distal end of a respective one of first and second half-belts, and received through a respective one of the second free buckles, the first and second tightening arranged to be superimposed on the lateral extensions of the plate against the patient's lumbar-abdominal area.

8. The lumbar support belt according to claim 7, wherein the plate includes a central opening for freeing the patient's posterior processes, the majority of said central opening extending along an axis designed to be positioned in the sagittal plane of the patient.

9. The lumbar support belt according to claim 7, wherein the dorsal plate comprises a scoring extending along an axis arranged to be positioned in the sagittal plane of the patient.

10. The lumbar support belt according to claim 7, wherein the free ends of the tightening straps form a loop to receive one of the patient's fingers, so as to facilitate the exertion of a pulling force on the tightening straps by the patient.

11. The lumbar support belt according to claim 7, wherein the proximal ends of the first and second half-belts each include at least one loop inside which a lateral extension of the dorsal plate is received, so as to allow sliding of the first and second half-belts with the dorsal plate.

12. The lumbar support belt according to claim 7, wherein the belts includes first and second elastic return means connecting the dorsal plate and the first or second half-belts, respectively, so as to continuously keep the plate and the first and second half-belts centered with respect to one another.
